(19) United States
(12) Patent Application Publication
(10) Pub. No.: US 2007/0099928 A1
(43) Pub. Date: May 3, 2007

(54) Amine salts of (-)-2-{2-(4-hydroxyphenyl) ethyl]thio} -3-4-(2-{4-(methylsulfonyl) oxy phenyl propanoic acid and their use in medicine

(76) Inventors: Matti Ahlqvist, Molndal (SE); Mikael Ulf Johan Dahlstrom, Molndal (SE); Bengt Ohlsson, Molndal (SE); Richard Anthony Storey, Macclesfield (GB); Nigel Phillip Taylor, Macclesfield (GB); Rebecca Woods, Macclesfield (GB)

Correspondence Address: MORGAN LEWIS & BOCKIUS LLP, 1111 PENNSYLVANIA AVENUE NW, WASHINGTON, DC, 20004, US

(21) Appl. No.: 10/561,222

(22) PCT Filed: Jun. 16, 2004
(86) PCT No.: PCT/GB04/02576

§ 371 Date: Dec. 19, 2005

(30) Foreign Application Priority Data
Jun. 18, 2003 (GB) 0314130.6

(51) Int. Cl. A61K 31/495; A61K 31/192; A61K 31/205; C07C 313/22

(52) U.S. Cl. 514/252.12; 514/554; 514/570; 544/358; 562/429; 562/430

(57) ABSTRACT

A tert-butylamine salt, a piperazine salt, a choline salt, a tris(hydroxymethyl)methylamine salt, a lysine salt or an adamantylamine salt of (−)-2-{[2-(4-hydroxyphenyl)ethyl]thio}-3-[4(2-{4-[(methylsulfonyl)oxy]phenoxy}ethyl)phenyl]propanoic acid processes for their preparation, their use in treating clinical conditions including lipid disorders (dyslipidemias) whether or not associated with insulin resistance and other manifestations of the metabolic syndrome, and pharmaceutical compositions containing them.

### FIELD OF THE INVENTION

The present invention relates to a tert-butylamine salt, a piperazine salt, a choline salt, a tris(hydroxymethyl)methylamine salt, a lysine or an adamantylamine salt of (−)-2-{[2-(4-hydroxyphenyl)ethyl]thio}-3-[4-(2-{4-[(methylsulfonyl)oxy]phenoxy}ethyl)phenyl]propanoic acid, to processes for their preparation, to their use in treating clinical conditions including lipid disorders (dyslipidemias) whether or not associated with insulin resistance and other manifestations of the metabolic syndrome, and to pharmaceutical compositions containing them

### BACKGROUND OF THE INVENTION

The metabolic syndrome including type 2 diabetes mellitus, refers to a cluster of manifestations including insulin resistance with accompanying hyperinsulinaemia, possibly type 2 diabetes mellitus, arterial hypertension, central (visceral) obesity, dyslipidaemia observed as deranged lipoprotein levels typically characterised by elevated VLDL (very low density lipoproteins), small dense LDL particles and reduced HDL (high density lipoprotein) concentrations and reduced fibrinolysis.

Recent epidemiological research has documented that individuals with insulin resistance run a greatly increased risk of cardiovascular morbidity and mortality, notably suffering from myocardial infarction and stroke. In type 2 diabetes mellitus atherosclerosis related conditions cause up to 80% of all deaths.

In clinical medicine there is awareness of the need to increase the insulin sensitivity in patients with the metabolic syndrome and thus to correct the dyslipidaemia which is considered to cause the accelerated progress of atherosclerosis. However, currently this is not a universally accepted diagnosis with well-defined pharmacotherapeutic indications.

Co-pending PCT application No. PCT/GB02/05743 discloses compounds of formula A 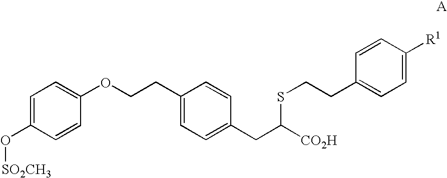
wherein R¹ represents chloro, fluoro or hydroxy as well as optical isomers and racemates thereof as well as pharmaceutically acceptable salts, prodrugs, solvates and crystalline forms thereof which are selective PPARα modulators (for a review of the PPARs (peroxisome proliferator-activated receptors ) see T. M. Willson et al, J Med Chem 2000, Vol 43, 527). These compounds are effective in treating conditions associated with insulin resistance. Specific pharmaceutically-acceptable salts of compounds of the formula A are not disclosed in PCT/GB02/05743. Further, no information is provided in relation to how crystalline forms of compounds of the formula A, and particularly salts thereof, may be prepared. The (−) enantiomer of the compound in which R¹ represents hydroxy is prepared as the free acid in this application. However, this compound is a thick oil with a syrup-like consistency and thus is not suitable for use in pharmaceutical formulations. Therefore there exists a need for a solid form of this compound which has physical and chemical properties suitable for use in pharmaceutical formulations. Many salts were tried but most of these either could not be formed in the solid state or were amorphous with a low glass transition temperature. Salts with suitable properties for pharmaceutical formulation have now been found.

In the formulation of drug compositions, it is important for the drug substance to be in a form in which it can be conveniently handled and processed. This is of importance, not only from the point of view of obtaining a commercially viable manufacturing process, but also from the point of view of subsequent manufacture of pharmaceutical formulations comprising the active compound.

Further, in the manufacture of drug compositions, it is important that a reliable, reproducible and constant plasma concentration profile of drug is provided following administration to a patient.

Chemical stability, solid state stability, and “shelf life” of the active ingredients are also very important factors. The drug substance, and compositions containing it, should preferably be capable of being effectively stored over appreciable periods of time, without exhibiting a significant change in the active component's physico-chemical characteristics (e.g. its chemical composition, density, hygroscopicity and solubility).

Moreover, it is also important to be able to provide the drug in a form which is as chemically pure as possible.

The skilled person will appreciate that, typically, if a drug can be readily obtained in a stable form, such as a stable crystalline form, advantages may be provided, in terms of ease of handling, ease of preparation of suitable pharmaceutical formulations, and a more reliable solubility profile.

### DESCRIPTION OF THE INVENTION

The present invention provides a tert-butylamine salt, a piperazine salt, a choline salt, a tris(hydroxymethyl)methylamine salt, a lysine salt or an adamantylamine a salt of (−)-2-{[2-(4-hydroxyphenyl)ethyl]thio}-3-[4-(2-{4-[(methylsulfonyl)oxy]phenoxy}-ethyl)phenyl]propanoic acid.

In particular the present invention provides a tert-butylamine salt, a piperazine salt, a choline salt or a tris(hydroxymethyl)methylamine salt of (−)-2-{[2-(4-hydroxyphenyl)ethyl]thio }-3-[4-(2-{4-[(methylsulfonyl)oxy]phenoxy}ethyl)phenyl]propanoic acid.

It will be understood that the present invention includes one or any combination of more than one of the above salts.

We have found that certain compounds of the invention have the advantage that they may be prepared in crystalline form.

According to a further aspect of the invention there is provided a compound of the invention in substantially crystalline form.

Although we have found that it is possible to produce compounds of the invention in forms which are greater than 80% crystalline, by “substantially crystalline” we include greater than 20%, preferably greater than 30%, and more preferably greater than 40% (e.g. greater than any of 50, 60, 70, 80 or 90%) crystalline.

According to a frter aspect of the invention there is also provided a compound of the invention in partially crystalline form. By “partially crystalline” we include 5% or between 5% and 20% crystalline.

The degree (%) of crystallinity may be determined by the skilled person using X-ray owder diffraction (XRPD). Other techniques, such as solid state NMR, FT-IR, Raman spectroscopy, differential scanning calorimetry (DSC) and microcalorimetry, may also be used.

Compounds of the invention, and particularly crystalline compounds of the invention, may have improved stability when compared to compounds disclosed in PCT/GB02/05743.

The term “stability” as defined herein includes chemical stability and solid state stability.

By “chemical stability”, we include that it may be possible to store compounds of the invention in an isolated form, or in the form of a formulation in which it is provided in admixture with pharmaceutically acceptable carriers, diluents or adjuvants (e.g. in an oral dosage form, such as a tablet, capsule etc.), under normal storage conditions, with an insignificant degree of chemical degradation or decomposition.

By “solid state stability”, we include that it may be possible to store compounds of the invention in an isolated solid form, or in the form of a solid formulation in which it is provided in admixture with pharmaceutically acceptable carriers, diluents or adjuvants (e.g. in an oral dosage form, such as a tablet, capsule etc.), under normal storage conditions, with an insignificant degree of solid state transformation (e.g. crystallisation, recrystallisation, solid state phase transition, hydration, dehydration, solvatisation or desolvatisation).

Examples of “normal storage conditions” include temperatures of between minus 80 and plus 50° C. (preferably between 0 and 40° C. and more preferably room temperatures, such as 15 to 30° C.), pressures of between 0.1 and 2 bars (preferably at atmospheric pressure), relative humidities of between 5 and 95% (preferably 10 to 60%), and/or exposure to 460 lux of UV/visible light, for prolonged periods (ie. greater than or equal to six months). Under such conditions, compounds of the invention may be found to be less than 15%, more preferably less than 10%, and especially less than 5%, chemically degraded/decomposed, or solid state transformed, as appropriate. The skilled person will appreciate that the above-mentioned upper and lower limits for temperature, pressure and relative humidity represent extremes of normal storage conditions, and that certain combinations of these extremes will not be experienced during normal storage (e.g. a temperature of 50° C. and a pressure of 0.1 bar).

It may be possible to crystallise salts of compounds of formula A with or without the presence of a solvent system (e.g. crystallisation may be from a melt, under supercritical conditions, or achieved by sublimation). However, we prefer that crystallisation occurs from an appropriate solvent system.

According to a further aspect of the invention, there is provided a process for the preparation of a crystalline compound of the invention which comprises crystallising a compound of the invention from an appropriate solvent system.

Crystallisation temperatures and crystallisation times depend upon the salt that is to be crystallised, the concentration of that salt in solution, and the solvent system that is used.

Crystallisation may also be initiated and/or effected by way of standard techniques, for example with or without seeding with crystals of the appropriate crystalline compound of the invention.

Different crystalline forms of the compounds of the invention may be readily characterised using X-ray powder diffraction (XRPD) methods, for example as described hereinafter.

In order to ensure that a particular crystalline form is prepared in the absence of other crystalline forms, crystallisations are preferably carried out by seeding with nuclei and/or seed crystals of the desired crystalline form in substantially complete absence of nuclei and/or seed crystals of other crystalline forms. Seed crystals of appropriate compound may be prepared, for example, by way of slow evaporation of solvent from a portion of solution of appropriate salt.

Compounds of the invention may be isolated using techniques which are well known to those skilled in the art, for example decanting, filtering or centrifuging.

Compounds may be dried using standard techniques.

Further purification of compounds of the invention may be effected using techniques, which are well known to those skilled in the art. For example impurities may be removed by way of recrystallisation from an appropriate solvent system. Suitable temperatures and times for the recrystallisation depend upon the concentration of the salt in solution, and upon the solvent system that is used.

When compounds of the invention are crystallised, or recrystallised, as described herein, the resultant salt may be in a form which has improved chemical and/or solid state stability, as mentioned hereinbefore.

Compounds of the invention have the advantage that they may be more efficacious, be less toxic, be longer acting, have a broader range of activity, be more potent, produce fewer side effects, be more easily absorbed, and/or have a better pharmacokinetic profile (e.g. higher oral bioavailability and/or lower clearance), than, and/or have other useful pharmacological, physical, or chemical, properties over, compounds known in the prior art. Compounds of the invention may have the further advantage that they may be administered less frequently than compounds known in the prior art.

Compounds of the invention may also have the advantage that they are in a form which provides for improved ease of handling. Further, compounds of the invention have the advantage that they may be produced in forms which may have improved chemical and/or solid state stability (including e.g. due to lower hygroscopicity). Thus, such compounds of the invention may be stable when stored over prolonged periods.

Compounds of the invention may also have the advantage that they may be crystallised in good yields, in a high purity, rapidly, conveniently, and at a low cost.

These salts have activity as medicaments, in particular the salts are selective agonists of PPARα, that is, their EC₅₀ for PPARα is at least ten times lower than their EC₅₀ for PPARγ wherein the EC₅₀s are measured and calculated as described in the assays later in this document. The compounds are potent and selective.

It will be understood by those skilled in the art that where (−) occurs in this specification that the acid has a negative rotation when measured using the conditions and concentration described in the experimental section. It should be understood that the salts of the present invention may have (+) rotation provided that the absolute configuration of the salt is the same as the configuration of the (−)-parent acid.

It will also be understood that the compounds of the present invention may exist in solvated, for example hydrated, as well as unsolvated forms. It is to be understood that the present invention encompasses all such solvated and unsolvated forms.

Methods of Preparation

The compound of the invention may be prepared as outlined below. However, the invention is not limited to these methods.

The salts may be prepared by reacting (−)-2-{[2-(4-hydroxyphenyl)ethyl]thio}-3-[4-(2-{4-[(methylsulfonyl)oxy]phenoxy}ethyl)phenyl]propanoic acid with the appropriate amine, for example tert-butylamine, piperazine, choline, tris(hydroxymethyl)methylamine, lysine or adamantanylamine in an inert solvent, for example ethanol, methanol, propan-2-ol, ethyl acetate, toluene or mixtures thereof or a mixture of ethanol or methanol or propan-2-ol and water, at a temperature in the range of 0-100° C. and isolating the solid salt. The salt may be isolated by cooling the reaction solution and optionally seeding the solution with the desired product and/or concentrating the solution. Optionally the product may be isolated by adding an antisolvent to a solution of the product in an inert solvent. The solid may be collected by methods known to those skilled in the art for example filtration or centrifugation. (−)-2-{[2-(4-Hydroxyphenyl)ethyl]thio}-3-[4-(2-{4-[(methylsulfonyl)oxy]phenoxy}-ethyl)phenyl]propanoic acid may be prepared as described in the Examples.

The expression “inert solvent” refers to a solvent which does not react with the starting materials, reagents, intermediates or products in a manner which adversely affects the yield of the desired product.

In another aspect the present invention provides the compound obtainable by reacting (−)-2-{[2-(4-hydroxyphenyl)ethyl]thio}-3-[4-(2-{4-[(methylsulfonyl)oxy]phenoxy}-ethyl)phenyl]propanoic acid and tert-butylamine in ethanol and isolating the product. Particularly an equivalent of tert-butylamine is used.

In another aspect the present invention provides the compound obtainable by reacting (−)-2-{[2-(4-hydroxyphenyl)ethyl]thio}-3-[4-(2-{4-[(methylsulfonyl)oxy]phenoxy}-ethyl)phenyl]propanoic acid and piperazine in ethyl acetate and isolating the product. Particularly an equivalent of piperazine is used.

In another aspect the present invention provides the compound obtainable by reacting (−)-2-{[2-(4-hydroxyphenyl)ethyl]thio}-3-[4-(2-{4-[(methylsulfonyl)oxy]phenoxy}-ethyl)phenyl]propanoic acid and piperazine in toluene and isolating the product. Particularly an equivalent of piperazine is used.

In another aspect the present invention provides the compound obtainable by reacting (−)-2-{[2-(4-hydroxyphenyl)ethyl]thio}-3-[4-(2-{4-[(methylsulfonyl)oxy]phenoxy}-ethyl)phenyl]propanoic acid and choline in an inert solvent and isolating the product. Particularly an equivalent of choline is used.

In another aspect the present invention provides the compound obtainable by reacting (−)-2-{[2-(4-hydroxyphenyl)ethyl]thio}-3-[4-(2-{4-[(methylsulfonyl)oxy]phenoxy}-ethyl)phenyl]propanoic acid and tris(hydroxymethyl)methylamine in an inert solvent for example ethanol and/or acetone and isolating the product. Particularly an equivalent of tris(hydroxymethyl)methylamine is used.

In another aspect the present invention provides the compound obtainable by reacting (−)-2-{[2-(4-hydroxyphenyl)ethyl]thio}-3-[4-(2-{4-[(methylsulfonyl)oxy]phenoxy}-ethyl)phenyl]propanoic acid and lysine in an inert solvent, for example methanol, ethanol, propan-2-ol or water or mixtures thereof, and isolating the product. Particularly an equivalent of lysine is used.

In another aspect the present invention provides the compound obtainable by reacting (−)-2-{[2-(4-hydroxyphenyl)ethyl]thio}-3-[4-(2-{4-[(methylsulfonyl)oxy]phenoxy}-ethyl)phenyl]propanoic acid and adamantylamine in an inert solvent and isolating the product. Particularly an equivalent of adamantylamine is used. The invention also provides the following embodiments.

A tert-butylamine salt (−)-2-{[2-(4-hydroxyphenyl)ethyl]thio}-3-[4-(2-{4-[(methylsulfonyl)oxy]phenoxy}ethyl)phenyl]propanoic acid characterised by an X-ray powder diffraction pattern characterised by peaks with d-values at 10.1, 5.9, 5.3. 4.66 and 4.09 Å.

A tert-butylamine salt (−)-2-{[2-(4-hydroxyphenyl)ethyl]thio}-3-[4-(2-{4-[(methylsulfonyl)oxy]phenoxy}ethyl)phenyl]propanoic acid having the XRPD pattern substantially as disclosed in figure B.

A piperazine salt of (−)-2-{[2-(4-hydroxyphenyl)ethyl]thio}-3-[4-(2-{4-[(methylsulfonyl)oxy]phenoxy}ethyl)phenyl]propanoic acid characterised by an X-ray powder diffraction pattern characterised by peaks with d-values at 12.2, 5.2, 4.67, 4.23 and 3.99 Å.

A piperazine salt of (−)-2-{[2-(4-hydroxyphenyl)ethyl]thio}-3-[4-(2-{4-[(methylsulfonyl)oxy]phenoxy}ethyl)phenyl]propanoic acid having the XRPD pattern substantially as disclosed in figure A.

A tris(hydroxymethyl)methylamine salt of (−)-2-{[2-(4-hydroxyphenyl)ethyl]thio}-3-[4-(2-{4-[(methylsulfonyl)oxy]phenoxy}ethyl)phenyl]propanoic acid characterised by an X-ray powder diffraction pattern characterised by peaks with d-values 14.7, 7.4, 4.8, 4.3 and 3.7 Å.

A tris(hydroxymethyl)methylamine salt of (−)-2-{[2-(4-hydroxyphenyl)ethyl]thio}-3-[4-(2-{4-[(methylsulfonyl)oxy]phenoxy}ethyl)phenyl]propanoic acid having the XRPD pattern substantially as disclosed in figure C.

A lysine salt of (−)-2-{[2-(4-hydroxyphenyl)ethyl]thio}-3-[4-(2-{4-[(methylsulfonyl)oxy]phenoxy}ethyl)phenyl]propanoic acid characterised by an X-ray powder diffraction pattern characterised by peaks with d-values 5.4, 5.0, 4.5, 4.3 and 4.0 Å.

A lysine salt of (−)-2-{[2-(4-hydroxyphenyl)ethyl]thio}-3-[4-(2-{4-[(methylsulfonyl)oxy]phenoxy}ethyl)phenyl]propanoic acid having the XRPD pattern substantially as disclosed in figure D.

A lysine salt of (−)-2-{[2-(4-hydroxyphenyl)ethyl]thio}-3-[4-(2-{4-[(methylsulfonyl)oxy]phenoxy}ethyl)phenyl]propanoic acid characterised by an X-ray powder diffraction pattern characterised by peaks with d-values 21.3, 12.9, 7.7, 7.1 and 4.7 Å.

A lysine of (−)-2-{[2-(4-hydroxyphenyl)ethyl]thio}-3-[4-(2-{4-[(methylsulfonyl)oxy]-phenoxy}ethyl)phenyl]propanoic acid having the XRPD pattern substantially as disclosed in figure E.

Pharmaceutical Preparations

The compound of the invention will normally be administered via the oral, parenteral, intravenous, intramuscular, subcutaneous or in other injectable ways, buccal, rectal, vaginal, transdermal and/or nasal route and/or via inhalation, in the form of pharmaceutical preparations in a pharmaceutically acceptable dosage form. Depending upon the disorder and patient to be treated and the route of administration, the compositions may be administered at varying doses.

Suitable daily doses of the compound of the invention in therapeutical treatment of humans are about 0.0001-100 mg/kg body weight, preferably 0.001-10 mg/kg body weight.

Oral formulations are preferred particularly tablets or capsules which may be formulated by methods known to those skilled in the art to provide doses of the active compound in the range of 0.5 mg to 500 mg for example 1 mg, 3 mg, 5 mg, 10 mg, ²⁵ mg, 50 mg, 100 mg and 250 mg.

According to a further aspect of the invention there is thus provided a pharmaceutical formulation including the compound of the invention in admixture with pharmaceutically acceptable adjuvants, diluents and/or carriers.

Pharmacological Properties

A compound of the invention is useful for the prophylaxis and/or treatment of clinical conditions associated with inherent or induced reduced sensitivity to insulin (insulin resistance) and associated metabolic disorders (also known as metabolic syndrome). These clinical conditions will include, but will not be limited to, general obesity, abdominal obesity, arterial hypertension, hyperinsulinaemia, hyperglycaemia, type 2 diabetes and the dyslipidaemia characteristically appearing with insulin resistance. This dyslipidaemia, also known as the atherogenic lipoprotein profile, is characterised by moderately elevated non-esterified fatty acids, elevated very low density lipoprotein (VLDL) triglyceride rich particles, high Apo B levels, low high density lipoprotein (HDL) levels associated with low apo AI particle levels and high Apo B levels in the presence of small, dense, low density lipoproteins (LDL) particles, phenotype B.

A compound of the present invention is expected to be useful in treating patients with combined or mixed hyperlipidemias or various degrees of hypertriglyceridemias and postprandial dyslipidemia with or without other manifestations of the metabolic syndrome.

Treatment with the present compound is expected to lower the cardiovascular morbidity and mortality associated with atherosclerosis due to their antidyslipidaemic as well as antiinflammatory properties. The cardiovascular disease conditions include macro-angiopathies of various internal organs causing myocardial infarction, congestive heart failure, cerebrovascular disease and peripheral arterial insufficiency of the lower extremities. Because of its insulin sensitizing effect the compound iss also expected to prevent or delay the development of type 2 diabetes from the metabolic syndrome and diabetes of pregnancy. Therefore the development of long-term complications associated with chronic hyperglycaemia in diabetes mellitus such as the micro-angiopathies causing renal disease, retinal damage and peripheral vascular disease of the lower limbs are expected to be delayed. Furthermore the compound may be useful in treatment of various conditions outside the cardiovascular system whether or not associated with insulin resistance, like polycystic ovarian syndrome, obesity, cancer and states of inflammatory disease including neurodegenerative disorders such as mild cognitive impairment, Alzheimer's disease, Parkinson's disease and multiple sclerosis.

A compound of the present invention is expected to be useful in controlling glucose levels in patients suffering from type 2 diabetes.

The present invention provides a method of treating or preventing dyslipidemias, the insulin resistance syndrome and/or metabolic disorders (as defined above) comprising the administration of a compound of the present invention to a mammal (particularly a human) in need thereof.

The present invention provides a method of treating or preventing type 2 diabetes comprising the administration of an effective amount of a compound of the present invention to a mammal (particularly a human) in need thereof.

In a further aspect the present invention provides the use of a compound of the present invention as a medicament.

In a further aspect the present invention provides the use of a compound of the present invention in the manufacture of a medicament for the treatment of insulin resistance and/or metabolic disorders.

Combination Therapy

A compound of the invention may be combined with another therapeutic agent that is useful in the treatment of disorders associated with the development and progress of atherosclerosis such as hypertension, hyperlipidaemias, dyslipidaemias, diabetes and obesity. A compound of the invention may be combined with another therapeutic agent that decreases the ratio of LDL:HDL or an agent that causes a decrease in circulating levels of LDL-cholesterol. In patients with diabetes mellitus a compound of the invention may also be combined with therapeutic agents used to treat complications related to micro-angiopathies.

A compound of the invention may be used alongside other therapies for the treatment of metabolic syndrome or type 2 diabetes and its associated complications, these include biguanide drugs, for example metformin, phenformin and buformin, insulin (synthetic insulin analogues, amylin) and oral antihyperglycemics (these are divided into prandial glucose regulators and alpha-glucosidase inhibitors). An example of an alpha-glucosidase inhibitor is acarbose or voglibose or miglitol. An example of a prandial glucose regulator is repaglinide or is nateglinide.

In another aspect of the invention, the compound of formula I, or a pharmaceutically acceptable salt thereof, may be administered in association with a PPAR modulating agent. PPAR modulating agents include but are not limited to a PPAR alpha and/or gamma and/or delta agonist, or pharmaceutically acceptable salts, solvates, solvates of such salts or prodrugs thereof. Suitable PPAR alpha and/or gamma agonists, pharmaceutically acceptable salts, solvates, solvates of such salts or prodrugs thereof are well known in the art. These include the compounds described in WO 01/12187, WO 01/12612, WO 99/62870, WO 99/62872, WO 99/62871, WO 98/57941, WO 01/40170, WO 04/000790, WO 04/000295, WO 04/000294, WO 03/051822, WO 03/051821, WO 02/096863, WO 03/051826, WO 02/085844, WO 01/040172, J Med Chem, 1996, 39, 665, Expert Opinion on Therapeutic Patents, 10 (5), 623-634 (in particular the compounds described in the patent applications listed on page 634) and J Med Chem, 2000, 43, 527 which are all incorporated herein by reference. Particularly a PPAR alpha and/or gamma and/or delta agonist refers to muraglitazar (BMS 298585), rivoglitazone (CS-011), netoglitazone (MCC-555), balaglitazone (DRF-2593, NN-2344), clofibrate, fenofibrate, bezafibrate, gemfibrozil, ciprofibrate, pioglitazone, rosiglitazone, AVE-0847, AVE-8134, CLX-0921, DRF-10945, DRF-4832, LY-518674, LY-818, LY-929, 641597, GW-590735, GW-677954, GW-501516, MBX-102, ONO-5129, KRP-101, R-483 (BM131258), TAK-559 or TAK-654. Particularly a PPAR alpha and/or gamma and/or delta agonist refers to tesaglitazar ((S)-2-ethoxy-3-[4-(2-{4-methanesulphonyl-oxyphenyl}ethoxy)phenyl]propanoic acid) and pharmaceutically acceptable salts thereof.

In addition a compound of the invention may be used in conjunction with a sulfonylurea for example: glimepiride, glibenclamide (glyburide), gliclazide, glipizide, gliquidone, chloropropamide, tolbutamide, acetohexamide, glycopyramide, carbutamide, glibonuride, glisoxepid, glybuthiazole, glibuzole, glyhexamide, glymidine, glypinamide, phenbutamide, tolcylamide and tolazamide. Preferably the sulfonylurea is glimepiride or glibenclamide (glyburide). More preferably the sulfonylurea is glimepiride. The present invention includes administration of a compound of the present invention in conjunction with one, two or more existing therapies described in this combination section. The doses of the other existing therapies for the treatment of type 2 diabetes and its associated complications will be those known in the art and approved for use by regulatory bodies for example the FDA and may be found in the Orange Book published by the FDA. Alternatively smaller doses may be used as a result of the benefits derived from the combination. The present invention also includes a compound of the present invention in combination with a cholesterol-lowering agent. The cholesterol-lowering agents referred to in this application include but are not limited to inhibitors of HMG-CoA reductase (3-hydroxy-3-methylglutaryl coenzyme A reductase). Suitably the HMG-CoA reductase inhibitor is a statin selected from the group consisting of atorvastatin, bervastatin, cerivastatin, dalvastatin, fluvastatin, itavastatin, lovastatin, mevastatin, nicostatin, nivastatin, pravastatin and simvastatin, or a pharmaceutically acceptable salt, especially sodium or calcium, or a solvate thereof, or a solvate of such a salt. A particular statin is atorvastatin, or a pharmaceutically acceptable salt, solvate, solvate of such a salt or a prodrug thereof. A more particular statin is atorvastatin calcium salt. A particularly preferred statin is, however, a compound with the chemical name (E)-7-[4-(4-fluorophenyl)-6-isopropyl-2-[methyl(methylsulfonyl)-amino]-pyrimidin-5-yl](3R,5S)-3,5-dihydroxyhept-6-enoic acid, [also known as (E)-7-[4-(4-fluorophenyl)-6-isopropyl-2-[N-methyl-N-(methylsulfonyl)-amino]pyrimidin-5-yl](3R,5S)-3,5-dihydroxyhept-6-enoic acid] or a pharmaceutically acceptable salt or solvate thereof, or a solvate of such a salt. The compound (E)-7-[4-(4-fluorophenyl)-6-isopropyl-2-[methyl-(methylsulfonyl)-amino]-pyrimidin-5-yl](3R,5S)-3,5-dihydroxyhept-6-enoic acid, and its calcium and sodium salts are disclosed in European Patent Application, Publication No. EP-A-0521471, and in Bioorganic and Medicinal Chemistry, (1997), 5(2), 437-444. This latter statin is now known under its generic name rosuvastatin.

In the present application, the term “cholesterol-lowering agent” also includes chemical modifications of the HMG-CoA reductase inhibitors, such as esters, prodrugs and metabolites, whether active or inactive.

The present invention also includes a compound of the present invention in combination with a bile acid sequestering agent, for example colestipol or cholestyramine or cholestagel.

The present invention also includes a compound of the present invention in combination with an inhibitor of the ileal bile acid transport system (IBAT inhibitor).

Suitable compounds possessing IBAT inhibitory activity have been described, see for instance the compounds described in WO 93/16055, WO 94/18183, WO 94/18184, WO 96/05188, WO 96/08484, WO 96/16051, WO 97/33882, WO 98/07449, WO 98/03818, WO 98/38182, WO 99/32478, WO 99/35135, WO 98/40375, WO 99/35153, WO 99/64409, WO 99/64410, WO 00/01687, WO 00/47568, WO 00/61568, WO 00/62810, WO 01/68906, DE 19825804, WO 00/38725, WO 00/38726, WO 00/38727, WO 00/38728, WO 00/38729, WO 01/68906, WO 01/66533, WO 02/32428, WO 02/50051, EP 864 582, EP489423, EP549967, EP573848, EP624593, EP624594, EP624595 and EP624596 and the contents of these patent applications are incorporated herein by reference.

Further suitable compunds possessing IBAT inhibitory activity have been described in WO 94/24087, WO 98/56757, WO 00/20392, WO 00/20393, WO 00/20410, WO 00/20437, WO 01/34570, WO 00/35889, WO 01/68637, WO 02/08211, WO 03/020710, WO 03/022825, WO 03/022830, WO 03/022286, WO 03/091232, WO 03/106482, JP 10072371, U.S. Pat. No. 5,070,103, EP 251 315, EP 417 725, EP 869 121, EP 1 070 703 and EP 597 107 and the contents of these patent applications are incorporated herein by reference.

Particular classes of IBAT inhibitors suitable for use in the present invention are benzothiepines, and the compounds described in the claims, particularly claim 1, of WO 00/01687, WO 96/08484 and WO 97/33882 are incorporated herein by reference. Other suitable classes of IBAT inhibitors are the 1,2-benzothiazepines, 1,4-benzothiazepines and 1,5-benzothiazepines. A further suitable class of IBAT inhibitors is the 1,2,5-benzothiadiazepines.

One particular suitable compound possessing IBAT inhibitory activity is (3R,5R)-3-butyl-3-ethyl-1,1-dioxido-5-phenyl-2,3,4,5-tetrahydro-1,4-benzothiazepin-8-yl β-D-glucopyranosiduronic acid (EP 864 582). Other suitable IBAT inhibitors include one of:
1,1-dioxo-3,3-dibutyl-5-phenyl-7-methylthio-8-(N—{(R)-1′-phenyl-1′-[N′-(carboxymethyl)carbamoyl]methyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-(carboxymethyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N—{(R)-1′-phenyl-1′-[N′-(2-sulphoethyl)carbamoyl]methyl}carbamoylmethoxy)-2,3,4,5-tetrahydro-1,5-benzothiazepine;
1,1-dioxo-3-butyl-3-ethyl-5-phenyl-7-methylthio-8-(N—{(R)-1′-phenyl-1′-[N′-(2-sulphoethyl)carbamoyl]methyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-(2-sulphoethyl)carbamoyl]-4-hydroxybenzyl}carbamoylmethoxy)-2,3,4,5-tetrahydro-1,5-benzothiazepine;
1,1-dioxo-3-butyl-3-ethyl-5-phenyl-7-methylthio-8-(N—{(R)-α-(N′-(2-sulphoethyl)carbamoyl]-4-hydroxybenzyl}carbamoylmethoxy)-2,3,4,5-tetrahydro-1,5-benzothiazepine;
1,1-dioxo-3-butyl-3-ethyl-5-phenyl-7-methylthio-8-(N—{(R)-α-[N′-(2-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-(2-carboxyethyl)carbamoyl]-4-hydroxybenzyl}carbamoylmethoxy)-2,3,4,5-tetrahydro-1,5-benzothiazepine;
1,1-dioxo-3-butyl-3-ethyl-5-phenyl-7-methylthio-8-(N—{(R)-α-[N′-(5-carboxypentyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′-(2-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{α-[N′-(2-sulphoethyl)carbamoyl]-2-fluorobenzyl}carbamoylmethoxy)-2,3,4,5-tetrahydro-1,5-benzothiazepine;
1,1-dioxo-3-butyl-3-ethyl-5-phenyl-7-methylthio-8-(N—{(R)-α-[N′—(R)-(2-hydroxy-1-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N—{(R)-α-[N′—(R)-(2-hydroxy-1-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-{N—{(R)-α-(N′—{(R)-1-[N″—(R)-(2-hydroxy-1-carboxyethyl)carbamoyl]-2-hydroxyethyl}carbamoyl)benzyl]carbamoylmethoxy}-2,3,4,5-tetrahydro-1,5-benzothiazepine;
1,1-dioxo-3-butyl-3-ethyl-5-phenyl-7-methylthio-8-(N-{α-[N′-(carboxymethyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-(N-{α-[N′-((ethoxy)(methyl)phosphoryl-methyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-{N—[(R)-α-(N′-{2-[(hydroxy)(methyl)phosphoryl]ethyl}carbamoyl)benzyl]carbamoylmethoxy}-2,3,4,5-tetrahydro-1,5-benzothiazepine;
1,1-dioxo-3,3-dibutyl-5-phenyl-7-methylthio-8-(N—{(R)-α-(N′-(2-methylthio-1-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-{N—[(R)-α-(N′-{2-[(methyl)(ethyl)phosphoryl]ethyl}carbamoyl)-4-hydroxybenzyl]carbamoylmethoxy}-2,3,4,5-tetrahydro-1,5-benzothiazepine;
1,1-dioxo-3,3-dibutyl-5-phenyl-7-methylthio-8-{N—[(R)-α-(N′-{2-[(methyl)(hydroxy)phosphoryl]ethyl}carbamoyl)-4-hydroxybenzyl]carbamoylmethoxy}-2,3,4,5-tetrahydro-1,5-benzothiazepine;
1,1-dioxo-3,3-dibutyl-5-phenyl-7-methylthio-8-(N—{(R)-α-[(R)—N′-(2-methylsulphinyl-1-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methoxy-8-[N—{(R)-α-[N′-(2-sulphoethyl)carbamoyl]-4-hydroxybenzyl}carbamoylmethoxy]-2,3,4,5-tetrahydro-1,5-benzothiazepine;
1,1-dioxo-3,3-dibutyl-5-phenyl-7-methylthio-8-(N—{(R)-α-[N—((R)-1-carboxy-2-methylthio-ethyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N—((S)-1-carboxy-2-(R)-hydroxypropyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N—((S)-1-carboxy-2-methylpropyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N—((S)-1-carboxybutyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N—((S)-1-carboxypropyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N—((S)-1-carboxyethyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N—((S)-1-carboxy-2-(R)-hydroxypropyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N-(2-sulphoethyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N—((S)-1-carboxyethyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N—((R)-1-carboxy-2-methylthioethyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N—{(S)-1-[N—((S)-2-hydroxy-1-carboxyethyl)carbamoyl]propyl}carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N—((S)-1-carboxy-2-methylpropyl)carbamoyl]benzyl}carbamoylmethoxy)-2,3,4,5-tetrahydro-1,2,5-benzothiadiazepine;
1,1-Dioxo-3,3-dibutyl-5-phenyl-7-methylthio-8-(N—{(R)-α-[N—((S)-1-carboxypropyl)carbamoyl]-4-hydroxybenzyl}carbamoylmethoxy)-2,3,4,5-tetrahydro-1,2,5-benzothiadiazepine;
1,1-Dioxo-3,3-dibutyl-5-phenyl-7-methylthio-8-[N—((R/S)-α-{N-[1-(R)-2-(S)-1-hydroxy-1-(3,4-dihydroxyphenyl)prop-2-yl]carbamoyl}-4-hydroxybenzyl)carbamoylmethoxy]-2,3,4,5-tetrahydro-1,2,5-benzothiadiazepine;
1,1-Dioxo-3,3-dibutyl-5-phenyl-7-methylthio-8-(N—{(R)-α-[N-(2-(S)-3-(R)-4-(R)-5-(R)-2,3,4,5,6-pentahydroxyhexyl)carbamoyl]-4-hydroxybenzyl}carbamoylmethoxy)-2,3,4,5-tetrahydro-1,2,5-benzothiadiazepine; and
1,1-Dioxo-3,3-dibutyl-5-phenyl-7-methylthio-8-(N—{(R)-α-[N-(2-(S)-3-(R)-4-(R)-5-(R)-2,3,4,5,6-pentahydroxyhexyl)carbamoyl]benzyl}carbamoylmethoxy)-2,3,4,5-tetrahydro-1,2,5-benzothiadiazepine;
   or a pharmaceutically acceptable salt, solvate, solvate of such a salt or a prodrug thereof.

According to an additional further aspect of the present invention there is provided a combination treatment comprising the administration of an effective amount of a compound of the present invention optionally together with a pharmaceutically acceptable diluent or carrier, with the simultaneous, sequential or separate administration one or more of the following agents selected from:
a CETP (cholesteryl ester transfer protein) inhibitor, for example those referenced and described in WO 00/38725 page 7 line 22—page 10, line 17 which are incorporated herein by reference;
a cholesterol absorption antagonist for example azetidinones such as SCH 58235 and those described in U.S. Pat. No. 5,767,115 which are incorporated herein by reference;
a MTP (microsomal transfer protein) inhibitor for example those described in Science, 282, 751-54, 1998 which are incorporated herein by reference;
a nicotinic acid derivative, including slow release and combination products, for example, nicotinic acid (niacin), acipimox and niceritrol;
a phytosterol compound for example stanols;
probucol;
an omega-3 fatty acid for example Omacor™;
an anti-obesity compound for example orlistat (EP 129,748) and sibutramine (GB 2,184,122 and U.S. Pat. No. 4,929,629);
an antihypertensive compound for example an angiotensin converting enzyme (ACE) inhibitor, an angiotensin II receptor antagonist, an andrenergic blocker, an alpha andrenergic blocker, a beta andrenergic blocker for example metoprolol, a mixed alpha/beta andrenergic blocker, an andrenergic stimulant, calcium channel blocker, an AT-1 blocker, a saluretic, a diuretic or a vasodilator;
a CB1 antagonist or inverse agonist for example as described in WO01170700 and EP 65635; aspirin;
a Melanin concentrating hormone (MCH) antagonist;
a PDK inhibitor; or
modulators of nuclear receptors for example LXR, FXR, RXR, and RORalpha;
or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Particular ACE inhibitors or pharmaceutically acceptable salts, solvates, solvate of such salts or a prodrugs thereof, including active metabolites, which can be used in combination with a compound of the present invention include but are not limited to, the following compounds: alacepril, alatriopril, altiopril calcium, ancovenin, benazepril, benazepril hydrochloride, benazeprilat, benzoylcaptopril, captopril, captopril-cysteine, captopril-glutathione, ceranapril, ceranopril, ceronapril, cilazapril, cilazaprilat, delapril, delapril-diacid, enalapril, enalaprilat, enapril, epicaptopril, foroxymithine, fosfenopril, fosenopril, fosenopril sodium, fosinopril, fosinopril sodium, fosinoprilat, fosinoprilic acid, glycopril, hemorphin-4, idrapril, imidapril, indolapril, indolaprilat, libenzapril, lisinopril, lyciumin A, lyciumin B, mixanpril, moexipril, moexiprilat, moveltipril, muracein A, muracein B, muracein C, pentopril, perindopril, perindoprilat, pivalopril, pivopril, quinapril, quinapril hydrochloride, quinaprilat, ramipril, ramiprilat, spirapril, spirapril hydrochloride, spiraprilat, spiropril, spiropril hydrochloride, temocapril, temocapril hydrochloride, teprotide, trandolapril, trandolaprilat, utibapril, zabicipril, zabiciprilat, zofenopril and zofenoprilat. Preferred ACE inhibitors for use in the present invention are ramipril, ramiprilat, lisinopril enalapril and enalaprilat. More preferred ACE inhibitors for uses in the present invention are ramipril and ramiprilat.

Preferred angiotensin II antagonists, pharmaceutically acceptable salts, solvates, solvate of such salts or a prodrugs thereof for use in combination with a compound of the present invention include, but are not limited to, compounds: candesartan, candesartan cilexetil, losartan, valsartan, irbesartan, tasosartan, telmisartan and eprosartan. Particularly preferred angiotensin II antagonists or pharmaceutically acceptable derivatives thereof for use in the present invention are candesartan and candesartan cilexetil.

Therefore in an additional feature of the invention, there is provided a method for for the treatment of type 2 diabetes and its associated complications in a warm-blooded animal, such as man, in need of such treatment which comprises administering to said animal an effective amount of a compound of the present invention in simultaneous, sequential or separate administration with an effective amount of one the other compounds described in this combination section, or a pharmaceutically acceptable salt, solvate, solvate of such a salt or a prodrug thereof.

Therefore in an additional feature of the invention, there is provided a method of treating hyperlipidemic conditions in a warm-blooded animal, such as man, in need of such treatment which comprises administering to said anal an effective amount of a compound of the present invention in simultaneous, sequential or separate administration with an effective amount of one the other compounds described in this combination section or a pharmaceutically acceptable salt, solvate, solvate of such a salt or a prodrug thereof.

According to a further aspect of the invention there is provided a pharmaceutical composition which comprises a compound of the present invention, and one of the other compounds described in this combination section or a pharmaceutically acceptable salt, solvate, solvate of such a salt or a prodrug thereof, in association with a pharmaceutically acceptable diluent or carrier.

According to a further aspect of the present invention there is provided a kit comprising a compound of the present invention and one of the other compounds described in this combination section or a pharmaceutically acceptable salt, solvate, solvate of such a salt or a prodrug thereof.

According to a further aspect of the present invention there is provided a kit comprising:
a) a compound of the present invention in a first unit dosage form;
b) one of the other compounds described in this combination section or a pharmaceutically acceptable salt, solvate, solvate of such a salt or a prodrug thereof; in a second unit dosage form; and
c) container means for containing said first and second dosage forms.

According to a further aspect of the present invention there is provided a kit comprising:
a) a compound of the present invention together with a pharmaceutically acceptable diluent or carrier, in a first unit dosage form;
b) one of the other compounds described in this combination section or a pharmaceutically acceptable salt, solvate, solvate of such a salt or a prodrug thereof, in a second unit dosage form; and
c) container means for containing said first and second dosage forms.

According to another feature of the invention there is provided the use of a compound of the present invention of the present invention and one of the other compounds described in this combination section, or a pharmaceutically acceptable salt, solvate, solvate of such a salt or a prodrug thereof, in the manufacture of a medicament for use in the the treatment of metabolic syndrome or type 2 diabetes and its associated complications in a warm-blooded animal, such as man.

According to another feature of the invention there is provided the use of a compound of the present invention and one of the other compounds described in this combination section, or a pharmaceutically acceptable salt, solvate, solvate of such a salt or a prodrug thereof, in the manufacture of a medicament for use in the treatment of hyperlipidaemic conditions in a warm-blooded animal, such as man.

According to a further aspect of the present invention there is provided a combination treatment comprising the administration of an effective amount of a compound of the present invention optionally together with a pharmaceutically acceptable diluent or carrier, with the simultaneous, sequential or separate administration of an effective amount of one of the other compounds described in this combination section, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Experimental

¹H NMR and ¹³C NMR measurements were performed on a Varian Mercury 300 or Varian UNITY plus 400, 500 or 600 spectrometers, operating at ¹H frequencies of 300, 400, 500 and 600 MHz, respectively, and at ¹³C frequencies of 75, 100, 125 and 150 MHz, respectively. Measurements were made on the delta scale (δ).

Unless otherwise stated, chemical shifts are given in ppm with the solvent as internal standard.

X-ray powder diffraction analysis (XRPD) was performed using variable slits on samples prepared according to standard methods without using any internal standard. Examples of standard methods used are those described in Giacovazzo, C. et al (1995), Fundamentals of Crystallography, Oxford University Press; Jenkins, R. and Snyder, R. L. (1996), Introduction to X-Ray Powder Diffractometry, John Wiley & Sons, New York; Bunn, C. W. (1948), Chemical Crystallography, Clarendon Press, London; or Klug, H. P. & Alexander, L. E. (1974), X-ray Diffraction Procedures, John Wiley and Sons, New York. X-ray analyses were performed using Cu-radiation on a Siemens D5000 diffractometer or a Philips X'Pert MPD. The X-axis in the figures below is 2-theta and the Y axis is intensity.

Differential scanning calorimetry (DSC) was performed using a Mettler DSC820, a Mettler DSC820E or a Perkin Elmer DSC 7 instrument, according to standard methods, for example those described in Höhne, G. W. H. et al (1996), Differential Scanning Calorimetry, Springer, Berlin.

Thermo-gravimetric analysis (TGA) was performed using a Mettler Toledo TGA850, a Mettler Toledo TG851 or a Perkin Elmer TGA 7 instrument. A ramp rate of 10° C./min was used.

It will be appreciated by the skilled person that crystalline forms of compounds of the invention may be prepared by analogy with processes described herein and/or in accordance with the Examples below, and may show essentially the same XRPD diffraction patterns and/or DSC and/or TGA thermograms as those disclosed herein. By “essentially the same” XRPD diffraction patterns and/or DSC and/or TGA thermograms, we include those instances when it is clear from the relevant patterns and/or thermograms (allowing for experimental error) that essentially the same crystalline form has been formed. When provided, DSC onset temperatures may vary in the range ±5° C. (e.g. ±2° C.), and XRPD distance values may vary in the range ±2 on the last decimal place. It will be appreciated by the skilled person that XRPD intensities may vary when measured for essentially the same crystalline form for a variety of reasons including, for example, preferred orientation.

¹H NMR and ¹³C NMR measurements were performed on a Varian Mercury 300 or Varian UNITY plus 400, 500 or 600 spectrometers, operating at ¹H frequencies of 300, 400, 500 and 600 MHz, respectively, and at ¹³C frequencies of 75, 100, 125 and 150 MHz, respectively. Measurements were made on the delta scale (δ).

Unless otherwise stated, chemical shifts are given in ppm with the solvent as internal standard.

| Abbreviations | |
|---|---|
| IPA | propan-2-ol |
| DMSO | dimethyl sulfoxide |
| EtOAc | ethyl acetate |
| DMF | N,N-dimethylformamide |
| THF | tetrahydrofuran |
| MeCN | acetonitrile |
| MeOH | methanol |
| TFA | trifluoroacetic acid |
| NH₄OAc | ammmonium acetate |

| NMR Abbreviations | |
|---|---|
| t | triplet |
| s | singlet |
| d | doublet |
| q | quartet |
| m | multiplet |
| bs | broad singlet |

XRPD Abbreviations

| Intensity (rel %) | Definition |
|---|---|
| 25-100 | vs (very strong) |
| 10-25 | s (strong) |
| 3-10 | m (medium) |
| 1-3 | w (weak) |

TGA thermogravimetric analysis
DSC differential scanning calorimetry
XRPD X-ray powder diffraction
d-value the spacing between successive parallel hkl planes in a crystal lattice

### EXAMPLES

Preparation of Starting Material

### 2-{[2-(4-Hydroxyphenyl)ethyl]thio}-3-[4-(2-{4-[(methylsulfonyl)oxy]phenoxy}ethyl)phenyl]propanoic acid

### (i) Methyl 2-chloro-3-[4-(2-hydroxyethyl)phenyl]propanoate

2-(4-Aminophenyl)ethanol (11 g, 81 mmol) and 32 ml conc HCl was dissolved in acetone and cooled to 0° C. Sodium nitrite (5.6 g, 81 mmol) in 20 ml water was added dropwise. The temperature was kept under 0° C. After one hour, methyl acrylate (70 g, 808 mmol) and CuI (1.6 g, 8 mmol) were added (<0° C.). The reaction mixture was stirred at room temperature overnight.

The solvent was evaporated and water was added. The water phase was extracted three times with EtOAc, the organic phases were pooled and washed with water, dried (MgSO₄) and evaporated under reduced pressure. The crude product was purified by flash chromatography using a 65:35 mixture of EtOAc and heptane as eluent. Further purification by preparative HPLC (using a gradient of CH₃CN/5% CH₃CN-waterphase containing 0.1M NH₄OAc as eluent) gave 9.7 g product (yield 49%) as an oil.

¹HNMR (400 MHz, CDCl₃): 2.84 (t, 3H), 3.15 (dd, 1H), 3.35 (dd, 1H), 3.75 (s, 3H), 3.84 (t, 3H), 4.43 (t, 1H), 7.17 (d, 4H)

### (ii) Methyl 3-(4-{2-[4-(benzyloxy)phenoxy]ethyl}phenyl)-2-chloropropanoate

Triphenylphosphine (2.4 g, 9 mmol) was added to a solution of methyl 2-chloro-3-[4-(2-hydroxyethyl)phenyl]propanoate (2.1 g, 8.5 mmol) and 4-(benzyloxy)phenol (1.7 g, 8 mmol) in 20 ml toluene under nitrogen atmosphere. The solution was warmed to 55° C. and diisopropyl azodicarboxylate (1.8 g, 9 mmol) was added. The reaction mixture was stirred at 55° C. overnight.

The mixture was allowed to cool and the solvent was evaporated under reduced pressure. The crude product was purified by flash chromatography using a 80:20 mixture of heptane and EtOAc as eluent to yield 2.28 g of the desired product (yield 61%) as colourless crystals.

¹HNMR (400 MHz, CDCl₃): 3.05 (t, 2H), 3.16 (dd, 1H), 3.36 (dd, 1H), 3.75 (s, 3H), 4.12 (t, 2H), 4.45 (t, 1H), 5.01 (s, 2H), 6.82 (i, 2H), 6.90 (m, 2H), 7.13-7.27 (m, 4H), 7.29-7.47 (m, 5H).

### iii) Methyl 2-chloro-3-{4-[2-(4-hydroxyphenoxy)ethyl]phenyl}propanoate

Methyl 3-(4-{2-[4-(benzyloxy)phenoxy]ethyl}phenyl)-2-chloropropanoate (1.0 g, 2.4 mmol) and dimethyl sulfide (0.9 g, 14 mmol) was dissolved in 60 ml CH₂Cl₂. Boron trifluoride etherate (2.0 g, 14 mmol) was added droppwise to the stirred solution. The reaction mixture was stirrred for two days at room temperature. Another equivalent (0.4 g, 2.87 mmol) boron trifluoride etherate was added and the stirring was continued overnight.

Water was added. The phases were separated and the aqueous phase was extracted twice with CH₂Cl₂. The organic phases were pooled, washed (water, brine), dried (Na₂SO₄) and evaporated under reduced pressure. Further purification by preparative HPLC using a gradient of CH₃CN/5% CH₃CN-waterphase containing 0.1M NH₄OAc gave 0.55 g of the desired product (yield 52%) as an oil.

¹HNMR (400 MHz, CDCl₃): 3.04 (t, 2H), 3.16 (dd, 1H), 3.35 (dd, 1H), 3.75 (s, 3H), 4.10 (t, 2H), 4.40 (t, 1H), 6.75 (m, 4H), 7.12-7.29 (m, 4H).

### (iv) Methyl 2-chloro-3-[4-(2-{4-[(methylsulfonyl)oxy]phenoxy}ethyl)phenyl]propanoate

Methyl 2-chloro-3-{4-[2-(4-hydroxyphenoxy)ethyl]phenyl}propanoate (334 mg, 1.0 mmol) and triethylamine (303 mg, 3.0 mmol) was dissolved in 20 ml dichlormethane and cooled to −20° C. under nitrogen atmosphere. Methanesulfonyl chloride (114 mg, 1.0 mmol) was added dropwise. The mixture was allowed to reach room temperature. After 2 hours dichlormethane was added, the mixture was washed (water, brine), dried (Na₂SO₄) and evaporated under reduced pressure to yield 394 mg pure product (yield 96%).

¹HNMR (400 MHz, CDCl₃): 3.02-3.11 (m,5H), 3.15 (dd, 1H), 3.35 (dd,1H), 3.74 (s, 3H), 4.14 (t, 21), 4.44 (t, 1H), 5.29 (s, 21), 6.88 (d, 2H), 7.14-7.25 (m, 6H).

### (v) Methyl 2-({2-[4-(benzyloxy)phenyl]ethyl}thio)-3-[4-(2-{4-[(methylsulfonyl)oxy]phenoxy}ethyl)phenyl]propanoate

2-[4-(Benzyloxy)phenyl]ethanethiol (334 mg, 1.4 mmol), methyl 2-chloro-3-[4-(2-{4-[(methylsulfonyl)oxy]phenoxy}ethyl)phenyl]propanoate (394 mg, 0.95 mmol) and potassium carbonate (189 mg, 1.4 mmol) were dissolved in 14 ml dry DMF and stirred under nitrogen atmosphere at room temperature overnight.

The solvent was evaporated under reduced pressure and the residue was dissolved in toluene. The organic phase was washed (water, brine), dried (MgSO₄) and evaporated. Further purification by preparative HPLC using a gradient of CH₃CN/5% CH₃CN-waterphase containing 0.1M NH₄OAc gave 477 mg of the desired product (yield 75%).

¹HNMR (400 MHz, CDCl₃): 2.76-2.89 (m, 4H), 2.95 (dd, 1H), 3.09 (m,5H), 3.20 (dd, 1H), 3.53 (m, 1H), 3.70 (s, 3H), 4.15 (t, 2H), 5.06 (s, 2H), 6.91 (m, 4H), 7.07-7.24 (m, 8H), 7.31-7.48 (m, 5H).

### (vi) Methyl 2-{[2-(4-hydroxyphenyl)ethyl]thio}-3-[4-(2-{4-[(methylsulfonyl)oxy]phenoxy}ethyl)phenyl]propanoate

To a solution of methyl 2-({2-[4-(benzyloxy)phenyl]ethyl}thio)-3-[4-(2-{4-[(methylsulfonyl)oxy]phenoxy}ethyl)phenyl]propanoate (477 mg, 0.8 mmol) and 15 ml dichlormethane, dimethyl sulfide (239 mg, 3.8 mol) and boron trifluoride etherate (545 mg, 3.8 mmol) were added. After 18 hours of stirring water was added to the reaction. The phases were separated and the aqueous phase was extracted twice with dichlormethane. The organic phases were pooled, dried (MgSO₄) and evaporated under reduced pressure. 274 mg of the desired product (yield 67%) was obtained as an oil.

¹HNMR (400 MHz, CDCl₃): 2.70-2.85 (m, 4H), 2.91 (dd, 1H), 3.05 (t, 2H), 3.10 (s, 3H), 3.17 (dd, 1H), 3.49 (m, 1H), 3.68 (s, 3H), 4.13 (t, 2H), 6.72 (d, 2H), 6.87 (d, 2H), 6.99 (d, 2H), 7.10-7.22 (m, 6H)

### (vii) 2-{[2-(4-Hydroxyphenyl)ethyl]thio}-3-[4-(2-{4-[(methylsulfonyl)oxy]-phenoxy}ethyl)phenyl]propanoic acid

Methyl 2-{[2-(4-hydroxyphenyl)ethyl]thio}-3-[4-(2-{4-[(methylsulfonyl)oxy]phenoxy}-ethyl)phenyl]propanoate (105 mg, 0.2 mmol) was dissolved in 6.5 ml of a 7:1 mixture of THF and water and cooled on an ice-bath Lithium hydroxide (9.4 mg, 0.4 mmol ) was added. Water was added to the reaction mixture after 24 hours of stirring at room temperature. The THF was evaporated under reduced pressure and the residue was acidified with 1M hydrochloric acid. The water phase was extracted with EtOAc (x3), the organic phases were pooled, washed (water, brine), dried (MgSO₄) and evaporated. The crude product was purified using preparative HPLC (eluent: CH₃CN/5% CH₃CN-waterphase containing 0.1M NH₄OAc) to give 74 mg of the desired product (yield 97%) as an oil.

¹HNMR (400 MHz, CDCl₃): 2.68-2.95 (m, 5H), 3.05 (t, 2H), 3.10 (s, 3H), 3.17 (dd, 1H), 3.47 (m, 1H), 4.12 (t, 2H), 6.70 (d, 2H), 6.86 (d, 2H), 6.97 (d, 2H), 7.12-7.21 (m, 6H).

¹³CNMR (100 MHz, CDCl₃): 33.8, 35.1, 35.5, 37.2, 37.3, 48.1, 69.3, 115.6, 115.8, 123.3, 129.3, 129.4, 129.9, 132.3, 136.2, 136.9, 142.8, 154.4, 158.0, 177.2.

### (viii) (−)-2-{[2-(4-hydroxyphenyl)ethyl]thio}-3-[4-(2-{4-[(methylsulfonyl)oxy]phenoxy}ethyl)phenyl]propanoic acid

The racemate of 2-{[2-(4-hydroxyphenyl)ethyl]thio}-3-[4-(2-{4-[(methylsulfonyl)-oxy]phenoxy}ethyl)phenyl]propanoic acid was separated into its enantiomers using chiral chromatography. A Chiralpak AD JDB01+AS003 (336×100 mm id.) and ethanol/formic acid 100/0.01% was used as mobile phase. The racemate (9 g) was dissolved in ethanol and injected onto the column. The first eluting peak was collected and UV-detected. The product (4.1 g) was obtained with an enantiomeric purity >99%. The optical rotation was found to be [α]²⁰_{D}=−33° by dissolving the enantiomer in methanol to give a concentration of 0.64 g/100 ml. The optical rotation was measured at 20° C. using the sodium line at 589 nm

¹H NMR (500 MHz, CD₃OD): 7.17-7.22 (6H, m), 6.99 (2H, d), 6.94 (2H, d), 6.69 (2H, d), 4.17 (2H, t), 3.46 (1H, t), 3.16 (3H, s), 3.13 (1H, dd), 3.05 (2H, t), 2.69-2.88 (5H, m).

### Example 1

### (−)-2-{[2-(4-hydroxyphenyl)ethyl]thio}-3-[4-(2-{4-[(methylsulfonyl)oxy]-phenoxy}ethyl)phenyl]propanoic acid tert-butylamine salt

(−)-2-{[2-(4-hydroxyphenyl)ethyl]thio}-3-[4-(2-{4-[(methylsulfonyl)oxy]-phenoxy}ethyl)phenyl]propanoic acid (125 mg) was dissolved in ethanol (0.5 ml) at room temperature. Tert-butylamine was added (1 eq., 26 μl). The crystallisation started after approximately 40-50 minutes. The slurry was left overnight. Then, more ethanol was added (0.5 ml) and it was left for 30 minutes. Finally, the crystals were filtered off and washed with ethanol (0.2 ml) and dried in air for an hour. The product was a white dry crystalline powder (98 mg), which corresponds to a yield of approximately 68%.

### Example 2

### (−)-2-{[2-(4-hydroxyphenyl)ethyl]thio}-3-[4-(2-{4-[(methylsulfonyl)oxy]-phenoxy}ethyl)phenyl]propanoic acid piperazine salt

(−)-2-{[2-(4-hydroxyphenyl)ethyl]thio}-3-[4-(2-{4-[(methylsulfonyl)oxy]-phenoxy)ethyl)phenyl]propanoic acid (125 mg) was dissolved in ethyl acetate (0.5 ml) at room temperature. A solution of piperazine (1 molar equivalent) in ethyl acetate was added slowly. The product was collected by filtration to give a piperazine salt (−)-2-{[2-(4-hydroxyphenyl)ethyl]thio}-3-[4-(2-{4-[(methylsulfonyl)oxy]phenoxy}ethyl)phenyl]-propanoic acid.

### Example 3

### (−)-2-{[2-(4-hydroxyphenyl)ethyl]thio}-3-[4-(2-{4-[(methylsulfonyl)oxy]-phenoxy}ethyl)phenyl]propanoic acid piperazine salt

A repeat of example 2 but using toluene as the solvent gave a piperazine salt of (−)-2-{[2-(4-hydroxyphenyl)ethyl]thio}-3-[4-(2-{4-[(methylsulfonyl)oxy]phenoxy}ethyl)phenyl]-propanoic acid.

### Example 4

A solution of tris(hydroxymethyl)methylamine (459.6 mg) in ethanol (40 ml) was added dropwise, with stirring, to an ethanol solution (20 ml) of (−)-2-{[2-(4-hydroxyphenyl)ethyl-]thio}-3-[4-(2-{4-[(methylsulfonyl)oxy]-phenoxy}ethyl)phenyl]propanoic acid (1.96 g, 1.0 equivalents) at 25° C. The solution was stirred overnight and then allowed to evaporate over 24 h. A white solid crystallised. The white solid was slurried in acetone for 24 h and then the solvent was evaporated to dryness. The resulting crystalline white solid was a single polymorph of (−)-2-{[2-(4-hydroxyphenyl)ethyl]thio}-3-[4-(2-{4-[(methylsulfonyl)oxy]-phenoxy}ethyl)phenyl]propanoic acid tris(hydroxymethyl)methylamine, 1.0:1.0 salt.

### Example 5

A solution of anhydrous (L)-lysine (515.0 mg) in ethanol/methanol (60 ml, 40:20) was added dropwise, with stirring, to an ethanol solution (20 ml) of (−)-2-{[2-(4-hydroxyphenyl)ethyl]-thio}-3-[4-(2-{4-[(methylsulfonyl)oxy]-phenoxy}ethyl)phenyl]propanoic acid (1.82 g, 1.0 equivalents) at 25° C. The solution was stirred overnight and then allowed to evaporate over 24 h. A white solid crystallised. This was designated as Form 1 (−)-2-{[2-(4-hydroxyphenyl)-ethyl]thio}-3-[4-(2-{4-[(methylsulfonyl)oxy]-phenoxy}ethyl)phenyl]propanoic acid Lysine, 1.0:1.0 salt.

Form 1 (40 mg) was slurried in IPA/water (1.17 ml IPA, 0.18 ml water) for 3 days at 25° C. and then the white solid was filtered off and air dried. This white solid was designated as Form 2 (−)-2-{[2-(4-hydroxyphenyl)ethyl]thio}-3-[4-(2-{4-[(methylsulfonyl)oxy]-phenoxy}ethyl)phenyl]propanoic acid Lysine 1.0:1.0 salt.

### Example 6

(−)-2-{[2-(4-hydroxyphenyl)ethyl]thio}-3-[4-(2-{4-[(methylsulfonyl)oxy]-phenoxy}ethyl)phenyl]propanoic acid (0.42 g, 0.81 mmol) was dissolved in ethyl acetate (10 ml) in a round bottom flask. Adamantylamine (0. 123 g, 0.81 mmol) was dissolved in a small portion of methylene chloride (2 ml) and the solution was added to the round bottom flask.

The solvent was slowly evaporated at room temperature until one quarter of the solvent remained. The crystals was isolated by filtration and dried under vacuum to give 0.25 g, yield 46% of (−)-2-{[2-(4-hydroxyphenyl)ethyl]thio}-3-[4-(2-{4-[(methylsulfonyl)oxy]-phenoxy}ethyl)phenyl]propanoic acid adamantylamine salt.

Properties

### 1) Examples of properties of piperazine salt of (−)-2-{[2-(4-hydroxyphenyl)ethyl]thio}-3-[4-(2-{4-[(methylsulfonyl)oxy]phenoxy}ethyl)phenyl]propanoic acid

DSC showed an endotherm with an extrapolated onset temperature of ca. 105° C. TGA showed a weight loss of ca. 0.4% w/w between 24-95° C. DSC analysis repeated on purer sample may give a higher melting point. Crystals of the piperazine salt of (−)-2-{[2-(4-hydroxyphenyl)ethyl]thio}-3-[4-(2-{[4-[(methylsulfonyl)oxy]phenoxy}ethyl)phenyl]propanoic acid (obtained by way of the examples above and/or by other ways) were analyzed by XRPD and the results are tabulated below and are shown in Figure A.

[image]

Figure A, XRPD pattern of piperazine salt of (−)-2-{[2-(4-hydroxyphenyl)ethyl]thio}-3-[4-(2-{4-[(methylsulfonyl)oxy]phenoxy}ethyl)phenyl]propanoic acid

| d-value | intensity |
|---|---|
| (Angstrom) | (rel) |
| 20.6 | w |
| 12.2 | s |
| 10.3 | w |
| 6.8 | m |
| 6.1 | w |
| 5.4 | w |
| 5.3 | m |
| 5.2 | s |
| 4.86 | m |
| 4.67 | s |
| 4.60 | w |
| 4.37 | m |
| 4.29 | w |
| 4.23 | s |
| 4.06 | w |
| 3.99 | m |
| 3.57 | w |
| 3.45 | w |
| 3.12 | w |

### 2) Examples of properties of tert-butylamine salt of (−)-2-{[2-(4-hydroxyphenyl)ethyl]thiol}-3-[4-(2-{4-[(methylsulfonyl)oxy]-phenoxy}ethyl)phenyl]propanoic acid

DSC showed an endotherm with an extrapolated onset temperature of ca. 123° C. TGA showed a weight loss of ca. 0.5% w/w between 25-80° C. and ca. 2.5% w/w between 80-125° C. DSC analysis repeated on purer sample may give a higher melting point. Crystals of the tert-butylamine salt of (−)-2-{[2-(4-hydroxyphenyl)ethyl]thio}-3-[4-(2-{4-[(methylsulfonyl)oxy]phenoxy}ethyl)phenyl]propanoic acid (obtained by way of the examples above and/or by other ways) were analyzed by XRPD and the results are tabulated below and are shown in Figure B.

Figure B, XRPD pattern of tert-butylamine salt of (−)-2-{[2-(4-hydroxyphenyl)ethyl]thio}-3-[4-(2-{4-[(methylsulfonyl)oxy]phenoxy}ethyl)phenyl]propanoic acid

[image]

| d-value | intensity |
|---|---|
| (Angstrom) | (rel) |
| 12.6 | w |
| 10.6 | m |
| 10.1 | vs |
| 8.0 | w |
| 7.8 | w |
| 6.1 | w |
| 6.0 | w |
| 5.9 | s |
| 5.3 | vs |
| 5.04 | w |
| 4.86 | w |
| 4.66 | s |
| 4.38 | w |
| 4.29 | m |
| 4.20 | w |
| 4.09 | s |
| 4.04 | w |
| 3.99 | m |
| 3.92 | m |
| 3.89 | w |
| 3.81 | w |
| 3.73 | w |
| 3.59 | w |
| 3.44 | m |
| 3.36 | m |
| 3.27 | m |
| 3.10 | w |
| 2.93 | w |
| 2.88 | w |
| 2.79 | w |
| 2.24 | w |

Standard XRD Measurement Conditions for Figures C, D and B

Samples are spun at 30 rpm to improve counting statistics. X-rays are generated by a: ‘copper long-fine focus tube’ operated at 40 kV and 40 mA, wavelength of X-rays −1.5418 Å. The data for each sample are obtained using the standard scintillation detector. The collimated X-ray source was passed through an Automatic Variable Divergence Slit set at V20 (20 mm path-length) and the reflected radiation directed through a 2 mm anti-scatter slit and a 0.2 mm detector slit. Each sample is exposed for 4 seconds per 0.020° 2θ increment (continuous scan mode) over the range 2° to 40° 2θ in theta-theta mode. N.B. Running time for each sample is thus 2 hours 6 minutes 40 seconds. Note that the secondary soller slit is left in position.

### 3) Examples of properties of tris(hydroxymethyl)methylamine salt of (−)-2-{[2-(4-hydroxyphenyl)ethyl]thio}-3-[4-(2-{4-[(methylsulfonyl)oxy]phenoxy}ethyl)phenyl]propanoic acid

DSC:

DSC endotherm onset=115° C.
   TGA:
weight loss 25-100° C.=0.1% w/w
weight loss 100-165° C.=0.2-% w/w

[image]

Figure C, XRPD pattern of tris(hydroxymethyl)methylamine salt of (−)-2-{[2-(4-hydroxyphenyl)ethyl]thio}-3-[4-(2-{4-[(methylsulfonyl)oxy]phenoxy}ethyl)phenyl]propanoic acid

| d-value | intensity |
|---|---|
| (Angstrom) | (rel) |
| 14.7 | vs |
| 7.4 | vs |
| 6.0 | vs |
| 5.8 | s |
| 5.0 | vs |
| 4.8 | vs |
| 4.4 | s |
| 4.3 | vs |
| 4.0 | s |
| 3.9 | s |
| 3.7 | vs |

### 4) Examples of properties of lysine salt of (−)-2-{[2-(4-hydroxyphenyl)ethyl]thio}-3-[4-(2-{4-[(methylsulfonyl)oxy]phenoxy}ethyl)phenyl[propanoic acid

Form 1

DSC:

DSC endotherm onset=155° C.
   TGA:
weight loss 25-100° C.=0.6% w/w
weight loss 100-165° C.=<0.1% w/w

[image]

Figure D, XRPD pattern of Form 1 of lysine salt of (−)-2-{[2-(4-hydroxyphenyl)ethyl]thio}-3-[4-(2-{4-[(methylsulfonyl)oxy]phenoxy}ethyl)phenyl]propanoic acid

| d-value | intensity |
|---|---|
| (Angstrom) | (rel) |
| 5.4 | vs |
| 5.0 | vs |
| 4.5 | vs |
| 4.3 | vs |
| 4.1 | vs |
| 4.0 | vs |
| 3.7 | vs |
| 3.6 | vs |

Form 2
DSC:
DSC endotherm onset=159° C.
   TGA:
weight loss 25-100° C.=0.5% w/w
weight loss 100-165° C.=0.2% w/w

[image]

Figure E, XRPD pattern of Form 2 of lysine salt of (−)-2-{[2-(4-hydroxyphenyl)ethyl]thio}-3-[4-(2-{4-[(methylsulfonyl)oxy]phenoxy}ethyl)phenyl]propanoic acid.

| d-value | intensity |
|---|---|
| (Angstrom) | (rel) |
| 21.3 | vs |
| 12.9 | vs |
| 7.7 | vs |
| 7.1 | vs |
| 4.7 | vs |
| 4.2 | vs |

Biological Activity

The activity of the compounds of the invention is demonstrated in the assays described in WO03/051821.

1. A tert-butylamine salt, a piperazine salt, a choline salt, a tris(hydroxymethyl)methyl-amine salt, a lysine salt or an adamantylamine salt of (−)-2-{[2-(4-hydroxyphenyl)ethyl]thio}-3-[4-(2-{4-[(methylsulfonyl)oxy]phenoxy}ethyl)phenyl]propanoic acid.

2. A salt according to claim 1 selected from a tert-butylamine salt, a piperazine salt, a choline salt or a tris(hydroxymethyl)-methylamine salt of (−)-2-{[2-(4-hydroxyphenyl)ethyl]-thio}-3-[4-(2-{4-[(methylsulfonyl)oxy]phenoxy}ethyl)phenyl]propanoic acid.

3. A salt according to claim 1 which is a tert-butylamine salt.

4. A salt according to claim 1 which is a piperazine salt.

5. A salt according to claim 1 which is a choline salt.

6. A salt according to claim 1 which is a tris(hydroxymethyl)methylamine salt.

7. A salt according to claim 1 which is a lysine salt.

8. A salt according to claim 1 which is an adamantylamine salt.

9. A salt as claimed in any one of claims 1 to 8 which may be a solvate, a hydrate, a mixed solvate/hydrate, an ansolvate or an anhydrate.

10. A pharmaceutical formulation comprising a compound according to any one of claims 1 to 9 in admixture with pharmaceutically acceptable adjuvants, diluents and/or carriers.

11. A method of treating or preventing lipid disorders (dyslipidemia) whether or not associated with insulin resistance comprising the administration of a compound according according to any one of claims 1 to 9 to a mammal in need thereof.

12. The use of a compound according to any one of claims 1 to 9 in the manufacture of a medicament for the treatment of lipid disorders (dyslipidemia) whether or not associated with insulin resistance.

13. A method of treating or preventing type 2 diabetes comprising the administration of an effective amount of a compound according to any one of claims 1 to 9 to a mammal in need thereof.

14. A pharmaceutical composition comprising a compound according to any one of claims 1 to 9 combined with another therapeutic agent that is useful in the treatment of disorders associated with the development and progress of atherosclerosis such as hypertension, hyperlipidaemias, dyslipidaemias, diabetes and obesity.
